(19) United States
(12) Patent Application Publication
(10) Pub. No.: US 2026/0166207 A1
(43) Pub. Date: Jun. 18, 2026

(54) METHOD FOR OPERATING A DEVICE FOR EXTRACORPOREAL BLOOD TREATMENT, AND DEVICE FOR EXTRACORPOREAL BLOOD TREATMENT

(71) Applicant: B. Braun Avitum AG, Melsungen (DE)

(72) Inventors: Heiko Riemenschneider, Wagenfurth (DE); Reinhardt Peters, Erftstadt (DE)

(21) Appl. No.: 19/421,658

(22) Filed: Dec. 16, 2025

(30) Foreign Application Priority Data
Dec. 17, 2024 (DE) 10 2024 138 426.3

(51) Int. Cl. A61M 1/16; A61M 1/34

(52) U.S. Cl. CPC A61M 1/1603; A61M 1/3403; A61M 1/3413; A61M 1/3434; A61M 2202/0021; A61M 2202/0413; A61M 2205/10; A61M 2205/3331; A61M 2205/3379; A61M 2205/50

(57) ABSTRACT

A method is used to operate a device for extracorporeal blood treatment. The device can perform haemodialysis and hemodiafiltration. During hemodiafiltration, blood is delivered through a blood side of a dialyzer by an electric blood pump. Substituate is delivered by an electric substituate pump. A delivery volumetric flow of the electric substituate pump is adjusted in such a way that a current consumption of the electric blood pump remains within predefinable limit values.

### CROSS-REFERENCE TO RELATED APPLICATION

This application claims priority under 35 U.S.C. § 119 to German Application No. 10 2024 138 426.3, filed on Dec. 17, 2024, the content of which is incorporated by reference herein in its entirety.

### FIELD

The object of the present disclosure is to make available a method for operating a device for extracorporeal blood treatment and also to make available a device for extracorporeal blood treatment, which method and device permit simple and efficient operation of the device for extracorporeal blood treatment.

### BACKGROUND

As regards haemodialysis, which is known per se, reference is made to the relevant specialist literature. During hemodiafiltration, the following steps are performed: blood is delivered through a blood side of a dialyzer by means of an electric blood pump, substituate is delivered by means of an electric substituate pump, and a delivery volumetric flow of the electric substituate pump is adjusted in such a way that a current consumption of the electric blood pump remains within predefinable limit values. In other words, the current consumption of the electric blood pump is regulated to a predefinable setpoint value, wherein the manipulated variable of the regulation is the delivery volumetric flow of the electric substituate pump. The current consumption of the electric blood pump corresponds to the torque generated by means of the electric blood pump, with current and torque being considered and used as equivalents within the meaning of the present disclosure.

### SUMMARY

The method according to the present disclosure serves for operating a device for extracorporeal blood treatment, for example in the form of a dialysis machine. The method can be used to perform haemodialysis (HD) and hemodiafiltration (HDF).

In one embodiment, when the current consumption of the electric blood pump rises above an upper limit value, the delivery volumetric flow of the electric substituate pump is reduced. Correspondingly, when the current consumption of the electric blood pump drops below a lower limit value, the delivery volumetric flow of the electric substituate pump is increased. The reduction or increase in the delivery volumetric flow of the electric substituate pump can take place, for example, proportionally to an amount of a difference between the current consumption of the electric blood pump and the upper limit value or lower limit value. It is further possible that the reduction or increase in the delivery volumetric flow of the electric substituate pump takes place by a fixed amount, and so on.

In one embodiment, the substituate is fed to a blood circulation of a patient by means of post-dilution.

In one embodiment, the method is started with haemodialysis, and then continuous monitoring is performed to ascertain whether a secondary membrane has formed on a membrane of the dialyzer, and, as soon as the secondary membrane has formed on the membrane of the dialyzer, a switch is made to hemodiafiltration. With regard to monitoring and ascertaining the formation of a secondary membrane, reference is made to the relevant specialist literature, for example to EP 3 231 464 A1.

In one embodiment, a predefined cross rate is set after the switch to hemodiafiltration, after which a resulting current consumption of the electric blood pump is measured at the predefined cross rate, and finally the predefinable limit values are calculated according to the measured current consumption.

The cross rate is defined as the ratio between the ultrafiltration rate Quf and the plasma flow rate Qp, i.e. cross rate=Quf/Qp. The ultrafiltration rate Quf is set by the user of the dialysis machine and is critical as regards the amount of liquid that is withdrawn from the patient during the treatment. The ultrafiltration pump, which “sucks” the liquid through the membrane of the dialyzer, is responsible for the ultrafiltration rate. The plasma flow rate Qp corresponds to the blood flow rate of the blood pump, which transports the patient's blood to the dialyzer. A cross rate of 25% means, for example, that 25% of the liquid in the blood is withdrawn by the ultrafiltration. The higher the cross rate, the more liquid is withdrawn from the blood and the thicker the blood becomes, which can ultimately lead to blockage of the dialyzer.

In one embodiment, the upper limit value is predefined in a range between 115% and 135% of the measured current consumption, and/or the lower limit value is predefined as 0.8 times to 0.95 times the upper limit value.

The device for extracorporeal blood treatment, or the dialysis machine, has a dialyzer, an electric blood pump for delivering blood through a blood side of the dialyzer, an electric substituate pump for delivering substituate, and a controller which controls the electric blood pump and the electric substituate pump in such a way that the method according to the present disclosure is performed.

By means of the present disclosure, it is possible to improve the therapy result and at the same time facilitate the work of the personnel carrying out the treatment.

According to the present disclosure, the treatment profile of the dialyzer is evaluated via the current supply to the blood pump. The amount of substituate is automatically reduced when there are detectable increases in the blood pump current. Correspondingly, the amount of substituate is increased again as soon as the blood pump current has dropped again to a calculated base value plus x%, where the x% can be adapted specifically to the patient.

According to the present disclosure, there is an automatically delayed HDF start in the HDF treatment mode with post-dilution. According to the present disclosure, the change in current consumption of the arterial blood pump is measured and evaluated.

The blood pump current is measured at the start of therapy and used as a basis for keeping a limit value for example ca. 20% to 30% above this measured initial value.

If an incipient blockage of the dialyzer is detected through a corresponding increase in current, the substitution is reduced until the current drops below the limit value again.

To detect a blockage, the changes in the torques or in the motor drive current of the blood pump are measured and evaluated.

It is possible to predefine a fixed alarm limit value which must not be exceeded, for example a motor drive current which corresponds to a torque of >50 mNm. This value is normally ca. 20 mNm.

Furthermore, a value that sets in for example during the first 10 minutes of the treatment period is determined and an additional warning limit is fixed. This is for example ca. 30 mNm higher than the determined value of the first 10 minutes. A warning or alarm is output only if this limit value is continuously exceeded. Thus, starting current and starting torque are not taken into consideration in the monitoring.

The present disclosure prevents errors in use and minimizes the risk of a dialyzer becoming blocked as a result of excessively high cross rates during HDF. It is no longer necessary to ascertain when the secondary membrane has formed far enough, since the time of switching from HD to HDF and the amount of substituate can be calculated automatically.

The device for extracorporeal blood treatment assists a user and monitors the therapy automatically. Infused quantities can be displayed continuously, likewise the expected variables that can be achieved with the currently available setting.

A patient-related limit value of the motor current can be set individually at any time.

### BRIEF DESCRIPTION OF THE DRAWINGS

The present disclosure is described in detail below with reference to the drawings, in which:

FIG. 1 shows schematically a device for extracorporeal blood treatment; and

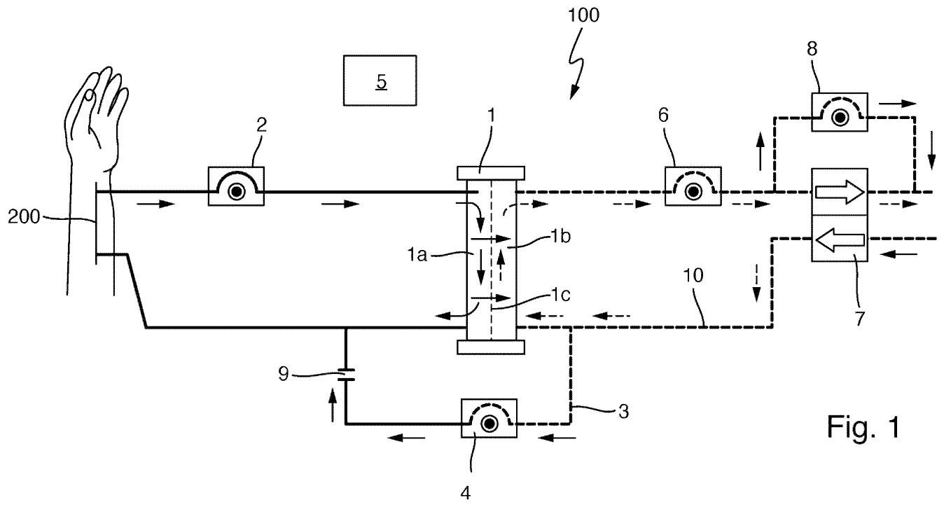

FIG. 2 shows a flowchart of a method according to the present disclosure for operating the device for extracorporeal blood treatment.

### DETAILED DESCRIPTION

The device 100 for extracorporeal blood treatment has: a dialyzer 1, an electric blood pump 2 for delivering blood through a blood side 1a of the dialyzer 1, an electric substituate pump 4 for delivering substituate 3, a dialysate pump 6 for delivering dialysate 10 through a dialysate side 1b of the dialyzer 1, a balancing device 7 and an ultrafiltration pump 8. With regard to the basic function of these components, reference is also made to the relevant specialist literature.

The device 100 for extracorporeal blood treatment also has a controller 5 which controls the electric blood pump 2 and the electric substituate pump 4 in such a way that a method as described below is performed.

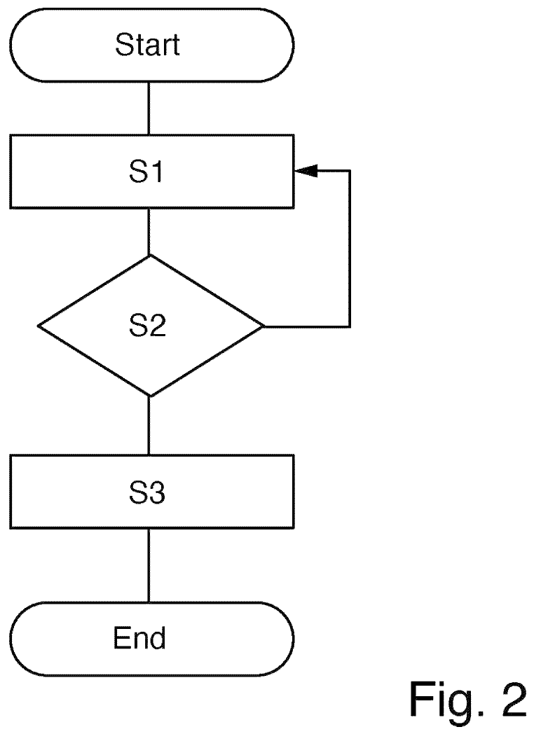

By means of the method, conventional haemodialysis (see step S1 in FIG. 2) and subsequent hemodiafiltration (see step S3 in FIG. 2) are performed.

The method begins with the haemodialysis in step S1.

As represented by the query S2, continuous monitoring is carried out to ascertain whether a secondary membrane has formed on a membrane 1c of the dialyzer 1. If no secondary membrane has formed, the haemodialysis is continued.

As soon as the secondary membrane has formed on the membrane 1c of the dialyzer 1, the method is continued in step S3 with hemodiafiltration.

During hemodiafiltration, the following steps are performed: blood is delivered through a blood side 1a of the dialyzer 1 by means of the electric blood pump 2, substituate 3 is delivered by means of the electric substituate pump 4, and a delivery volumetric flow of the electric substituate pump 4 is adjusted in such a way that a current consumption of the electric blood pump 2 remains within predefinable limit values.

When the current consumption of the electric blood pump 2 rises above an upper limit value, the delivery volumetric flow of the electric substituate pump 4 is reduced. When the current consumption of the electric blood pump 2 drops below a lower limit value, the delivery volumetric flow of the electric substituate pump 4 is increased (again).

The substituate 3 is fed to a blood circulation 200 of a patient by means of post-dilution 9.

A calculated cross rate is set after the switch to hemodiafiltration, and a resulting current consumption of the electric blood pump 2 is determined at the predefined cross rate. The upper limit value and the lower limit value are then determined according to the measured current consumption. For example, the upper limit value can be predefined in a range between 115% and 135% of the measured current consumption, and the lower limit value can be predefined as 0.8 times to 0.95 times the upper limit value.

1. A method for operating a device for extracorporeal blood treatment, the device configured to perform hemodiafiltration and haemodialysis, the method comprising the steps of:
delivering blood through a blood side of a dialyzer with an electric blood pump during hemodiafiltration;
delivering substituate via an electric substituate pump; and
adjusting a delivery volumetric flow of the electric substituate pump in such a way that a current consumption of the electric blood pump remains within predefinable limit values, the predefinable limit values comprising an upper limit value and a lower limit value.

2. The method according to claim 1, wherein:
when the current consumption of the electric blood pump rises above the upper limit value, the delivery volumetric flow of the electric substituate pump is reduced, and,
when the current consumption of the electric blood pump drops below the lower limit value, the delivery volumetric flow of the electric substituate pump is increased.

3. The method according to claim 1, wherein the substituate is fed to a blood circulation of a patient by post-dilution.

4. The method according to claim 1, further comprising the steps of:
performing haemodialysis with the device;
monitoring the dialyzer continuously to ascertain whether a secondary membrane has formed on a membrane of the dialyzer; and
switching to hemodiafiltration as soon as the secondary membrane has formed on the membrane of the dialyzer.

5. The method according to claim 4, further comprising the steps of:
setting a predefined cross rate after switching to hemodiafiltration;
measuring a current consumption of the electric blood pump at the predefined cross rate; and
calculating the predefinable limit values according to said current consumption.

6. The method according to claim 5, wherein
the upper limit value is predefined in a range between 115% and 135% of said current consumption, and/or
the lower limit value is predefined as 0.8 times to 0.95 times the upper limit value.

7. A device for extracorporeal blood treatment for performing the method according to claim 1, the device comprising:
a dialyzer;
an electric blood pump for delivering blood through a blood side of the dialyzer;
an electric substituate pump for delivering substituate; and
a controller that controls the electric blood pump and the electric substituate pump.
